(19) United States
(12) Patent Application Publication
(10) Pub. No.: US 2025/0128223 A1
(43) Pub. Date: Apr. 24, 2025

(54) REACTOR

(71) Applicant: JOHNSON MATTHEY DAVY TECHNOLOGIES LIMITED, London (GB)

(72) Inventors: Chris HEATON, LONDON (GB); Adrian LORD, LONDON (GB)

(21) Appl. No.: 18/835,142

(22) PCT Filed: Feb. 21, 2023
(86) PCT No.: PCT/GB2023/050380

§ 371 Date: Aug. 1, 2024

(30) Foreign Application Priority Data
Feb. 22, 2022 (GB) 2202363.4

(51) Int. Cl. B01J 8/04; C07C 7/163

(52) U.S. Cl. CPC B01J 8/0465; B01J 8/0492; C07C 7/163; B01J 2208/00548; B01J 2208/00893; B01J 2208/025; B01J 2208/027

(57) ABSTRACT

A liquid/gas reactor comprises: a primary catalyst bed having an inlet and outlet end; means for supplying a primary feed stream to the inlet end; a secondary catalyst bed having an inlet and outlet end; means for supplying a secondary feed stream to the inlet end; means for collecting at least partially converted liquid product from the outlet end of the primary catalyst bed and recycling at least a portion of the liquid product to the inlet end of the primary catalyst bed and secondary catalyst bed; a separating wall between the primary catalyst bed and secondary catalyst bed; means for supplying a primary gas stream only to the inlet end of the primary catalyst bed; and means for supplying a secondary gas stream only to the inlet end of the secondary catalyst bed. A process for carrying out a gas/liquid reaction using the reactor is also disclosed.

### TECHNICAL FIELD

The present disclosure relates to a liquid/gas reactor and a process for carrying out a gas/liquid reaction using said reactor. In particular, the disclosure relates to a liquid/gas reactor for carrying out a liquid recycle process, comprising primary and secondary catalyst beds.

### BACKGROUND

Chemical reactions between liquids and gases are often carried out over a solid catalyst bed. The reaction may be exothermic, i.e. generating heat, or it may be endothermic, i.e. utilising heat and effecting cooling of the surrounding atmosphere. In some reactions, the heat effects of the reaction are moderate; however, even in these, if the temperature is not controlled a loss of selectivity may result. For very exothermic or endothermic reactions, it is necessary to be more rigorous in controlling the heat effects. In extreme cases, the heat generated by strongly exothermic reactions can lead to thermal runaway. Similarly, the cooling effects of strongly endothermic reactions can lead to quenching of the reaction.

A commonly used method of controlling temperature in a liquid/gas reactor in which an exothermic or endothermic reaction occurs is to recycle heated or cooled product back into the reactor. This recycling has the effect of limiting the temperature rise, by diluting the reactant and allowing a lower conversion rate per pass-through. These so-called “liquid recycle” reactors are in widespread commercial use, for example, in the hydrogenation of benzene, the selective hydrogenations of olefins to remove alkynes and/or dienes, and in the hydrogenation of aldehydes to alcohols.

A typical reactor schematic for the selective hydrogenation of alkynes and dienes in C₂ and C₃ streams is illustrated in FIG. 1. The hydrogenation unit comprises a main reactor 1 and a finishing reactor 2. C₃ feedstock is supplied in line 3 to the main reactor 1 where it is reacted over a catalyst with hydrogen that has been supplied in line 4. Product is extracted in line 5 and cooled in cooler 6 before being passed in line 7 to liquid/gas separator 8. A proportion of the liquid is removed in line 9 and recycled to the main reactor 1. In the illustrated arrangement the recycle stream 9 is combined with the feedstock stream 3 before being supplied to the main reactor 1. The excess gas from the separator is removed in line 10.

The remaining liquid from the gas/liquid separator is removed in line 11 and fed to the finishing reactor 2. This reactor is a plug flow reactor. Product is removed in line 12. It will be understood that the liquid recycle reactor is partially back-mixed since the feed is diluted with the recycled product stream.

In the case of hydrogenation of 100% alkene to alkane or of aldehyde to alcohol, typical recycle rates of 10 to 20 times the feed rate are needed in order to avoid the temperature increase across the reactor exceeding 20° C. The recycled product significantly dilutes the reactant and thus the rate of reaction is lower and the heat of reaction is at least partially taken up by the recycled product, both of which help to reduce the temperature increase across the reactor.

The finishing reactor 2, which may also be known as a polishing reactor, is needed to make a high quality product which has low levels of unreacted feed components. In order to have liquid velocities suitable for good distribution, the cross-sectional area of the finishing stage has to be much smaller than the liquid recycle reactor and thus in order to achieve adequate catalyst volume a long finishing reactor must be used.

Another type of liquid recycle reactor, developed by the inventors of the present invention and disclosed in EP2516050B1, is illustrated in FIG. 2. The reactor disclosed in EP2516050B1 is a modification of a packed bed liquid/gas reactor 21 which improves the effectiveness of the catalyst and dispenses with the need for a finishing reactor, by providing a minor (or secondary) catalyst bed 23 extending through the bulk (or primary) catalyst bed 22. Separate feed streams 24, 25 are provided to the primary and secondary catalyst beds 22, 23, with a mixture of fresh feed 26 and recycled product 27 being supplied to the primary bed 22 while only recycled product 27 is supplied to the secondary bed 23, allowing an improved overall conversion to be achieved. A gas stream 28 is supplied to both the primary and secondary catalyst beds through a shared vapour space at the top of the reactor.

As the liquid and gaseous reactants pass through the catalyst beds, the pressure will drop from a maximum pressure at the inlet end of the catalyst beds (where reactants enter) to a minimum pressure at the outlet end (where products and excess reactants exit). The inventors of the present invention were surprised to find that, with the primary/secondary catalyst bed arrangement of the EP2516050B1 reactor, the pressure drop through the primary and secondary catalyst beds ideally needs to be identical in order to achieve an optimal overall conversion rate. The pressure drop can be calculated by the Ergun equation and is dependent on factors such as the particle size, bed voidage and liquid flow of each catalyst bed. Consistently achieving an identical pressure drop through both catalyst beds was found to be surprisingly difficult in practice. For example, the particle size of the catalyst in each bed might be different if loaded from different batches, or the void fraction in each bed might be different due to differences in loading of the catalyst or settling of the catalyst after loading.

The present invention is therefore aimed at solving one or more problems associated with the liquid/gas reactors of the prior art, and at providing a liquid/gas reactor capable of achieving a consistent and improved conversion rate.

### SUMMARY OF THE INVENTION

According to a first aspect of the invention, there is provided a liquid/gas reactor comprising:
(a) a primary catalyst bed having an inlet end and an outlet end;
(b) means for supplying a primary feed stream to the inlet end of the primary catalyst bed, the primary feed stream comprising fresh feed and recycled at least partially converted liquid product;
(c) a secondary catalyst bed having an inlet end and an outlet end, the secondary catalyst bed extending substantially vertically through the primary catalyst bed;
(d) means for supplying a secondary feed stream to the inlet end of the secondary catalyst bed, the secondary feed stream comprising recycled at least partially converted liquid product;
(e) means for collecting at least partially converted liquid product from the outlet end of the primary catalyst bed and recycling at least a portion of the at least partially converted liquid product to the inlet end of the primary catalyst bed and secondary catalyst bed;
(f) a separating wall between the primary catalyst bed and secondary catalyst bed;
(g) means for supplying a primary gas stream only to the inlet end of the primary catalyst bed; and
(h) means for supplying a secondary gas stream only to the inlet end of the secondary catalyst bed.

By providing means for supplying separate gas streams to the primary and secondary catalyst beds, the flow of gas to the primary and secondary catalyst beds are independent of each other and can optionally be individually controlled. The inventors were surprised to find that this allowed the effect of non-identical pressure drops across the catalyst beds to be mitigated, resulting in a more consistent and improved overall conversion rate.

Accordingly, in some embodiments the reactor comprises means for controlling the flowrate of the primary gas stream and the flowrate of the secondary gas stream individually. The flowrate of the gas streams may be controlled by any suitable means, such as by flow control valves. In one arrangement, the primary and secondary gas streams may be supplied to the reactor from separate sources with individually controlled flowrates. Alternatively, the primary and secondary gas streams may be supplied from a single source through a conduit which is branched to provide separate primary and secondary gas streams. In such embodiments, a flow control valve may be provided on each branch to control the flowrates of the primary and secondary gas streams individually.

The gas flowrate should be sufficient to keep the liquid feed saturated with gas reactant across the entire area of both catalyst beds. Saturation can, for example, be determined by the formation of bubbles of excess gas reactant. Alternatively or additionally hydrogen levels in a vent flow can be analysed; if the hydrogen level is sufficiently high it can be inferred that the liquid feed was saturated with gas reactant. If either bed is determined not to be saturated with gas reactant, the flowrate of gas to that bed may be increased.

The secondary catalyst bed is supplied only with feed which has already been subjected to reaction and is therefore at least partially converted. Consequently, the product stream exiting the secondary catalyst bed will provide a more fully converted final product than a reactor without the secondary catalyst bed.

The secondary catalyst bed may be disposed in any suitable location in the primary catalyst bed, although it will be understood that the secondary catalyst bed extends vertically through the primary catalyst bed so that the inlet and outlet ends of the secondary catalyst bed are not blocked by the primary catalyst bed. In some embodiments, the secondary catalyst bed is located in the centre of the primary catalyst bed, such that the primary catalyst bed forms an annulus around the secondary catalyst bed. Alternatively, the secondary catalyst bed may be offset to the side of the primary catalyst bed, or located against the wall of the reactor.

The reactor is a liquid/gas reactor in that fluid passing through the reactor is in both the liquid and the gas phase. The fluid passes over a solid, that is, heterogeneous, catalyst in the reactor.

The reactor and its components may be constructed of any suitable materials. In some embodiments, the separating wall is formed from an insulating material. This may be particularly useful where the primary and secondary catalyst beds are operated at different temperatures.

The separating wall may be of any suitable structure. For example, the separating wall may be formed by an internal pipe, in which the secondary catalyst bed is located. The separating wall may be of any suitable cross-sectional shape, such as circular. Alternatively, the separating wall may be formed by a half pipe fastened to the wall of the reactor, for example.

In some embodiments, the secondary catalyst bed comprises a cover to isolate the inlet end of the secondary catalyst bed from the inlet end of the primary catalyst bed, with the secondary gas and feed streams being supplied to the inlet end of the secondary catalyst bed inside the cover. It will be understood that the cover defines a closed cavity above the inlet end of the secondary catalyst bed, into which the secondary gas and feed streams are supplied. As such, the cover may be in the form of a convexly curved plate or a dome, or may comprise either sidewalls and a roof or a single continuous sidewall and a roof. In some embodiments, the cover at least partly comprises an extension of the separating wall above the primary and secondary catalyst beds, such that the extension of the separating wall forms the sidewall(s) of the cover.

In some embodiments, the cover comprises a removable cap. Providing a removable cap allows convenient access to the secondary catalyst bed when required, for example to replace the catalyst. In some embodiments, the cover further comprises a gasket for creating a gastight seal with the removable cap.

The reactor may comprise means for collecting a product stream from the outlet end of the secondary catalyst bed. In some embodiments, the means for collecting a product stream from the outlet end of the secondary catalyst bed includes a conduit for diverting the product stream from the secondary catalyst bed to a receiving portion of the reactor, the receiving portion being isolated, at least in terms of liquid flow, from the outlet end of the primary catalyst bed. The receiving portion may be a receptacle or a part of the reactor that is suitable for receiving the product stream exiting from the secondary catalyst bed and keeping it separate from the product stream exiting from the primary catalyst bed. For example, the receiving portion may comprise a baffle offset to one side of the bottom of the reactor, which acts as a weir flooded with product from the secondary catalyst bed. Alternatively, the baffle may be combined with a roof which the conduit passes through, to create a closed receptacle for receiving the product from the secondary catalyst bed; in such a case provision may be made for equalising vapour pressure between each side of the baffle and for overflow of product from the secondary catalyst bed to the outlet end of the primary catalyst bed.

In some embodiments, all of the at least partially converted product from the outlet end of the primary catalyst bed is recycled, with a portion being recycled to the inlet end of the primary catalyst bed and a portion being recycled to inlet end of the secondary catalyst bed.

In some embodiments, the reactor comprises means for adjusting the temperature of the recycled product stream, e.g. a heater and/or a cooler.

In some embodiments, the reactor comprises means for controlling the flowrate of the primary feed stream and the flowrate of the secondary feed stream individually. For example, the primary feed stream and the secondary feed stream may each be controlled by a flow control valve on their respective lines. The flowrate of the secondary feed stream supplied to the secondary catalyst bed may be equal to the final product rate. However, for ease of control, an excess of up to 100% is preferably supplied. The excess may, for example, be combined with the recycled stream from the primary catalyst bed. In a particularly preferred embodiment, the excess floods the weir and is thus combined with the output from the primary reactor bed. Preferably the final product rate is then controlled to maintain a desired liquid level on the primary catalyst bed side of the weir, by overflow of excess across the weir.

A feed stream ratio may be defined as the ratio of secondary feed stream flowrate to primary feed stream flowrate. Similarly, a bed cross-sectional area ratio may be defined as the ratio of secondary catalyst bed cross-sectional area to primary catalyst bed cross-sectional area.

Generally, the bed cross-sectional area ratio will be selected to preserve the required vapour/liquid mixing and achieve the required wetting of the catalyst. The bed cross-sectional area ratio may be from 0.1 to 5 times, from 0.2 to 3 times or from 0.5 to 2 times the feed stream ratio. In some embodiments, the bed cross-sectional area ratio is 1:1. This may allow the secondary catalyst bed to maintain a liquid velocity which gives good vapour/liquid mixing and good wetting, which will generally be of the same level as that achieved in the primary catalyst bed.

The recycle rate may be controlled so as to target a temperature rise of no more than 10-15° C. across the primary catalyst bed. The exact recycle rate required to achieve this will depend on the heat produced by the reaction, which is in turn dependent on the reactants used. For example, lighter alcohols may produce more reaction heat than heavier alcohols and thus require a higher recycle rate in order to increase the dilution factor. In general, the ratio of recycled stream to fresh feed supplied to the primary catalyst bed may be from 1 to 100, from 5 to 50, from 10 to 40, from 15 to 35, or from 20 to 30. A ratio of from 20 to 30 may be particularly preferred for reactions such as hydrogenation of butanal, for example. For octanal hydrogenation lower ratios can be used, both because the heat of reaction is lower and because the heavier alcohol is less sensitive to temperature and thus a higher temperature rise may be tolerated. A ratio of from 1 to 10 may for example be used. The ratio of recycled product supplied to the secondary catalyst bed to fresh feed supplied to the primary catalyst bed may be from 1 to 2. The ratio of recycled product supplied to the secondary catalyst bed to final product rate is preferably greater than 1, for example from 1 to 2.

Any suitable catalyst may be used in the primary and secondary catalyst beds. Generally, the selection of catalyst will depend on the reaction to be carried out, but may include nickel, copper, chromium, palladium, or any mixture thereof. The catalyst may also be of any suitable form, such as pellets, extrudates, resins or impregnated packing, for example. Suitable catalyst supports may, for example, include alumina, silica, vanadia, zirconia or carbon. The catalyst used in the primary and secondary catalyst beds may be the same or different.

The reactor may be suitable for use with any exothermic or endothermic reaction which can be carried out over a solid catalyst bed. Examples of exothermic reactions include hydrogenations of aldehydes, ketones, alkynes, dienes, or aromatic compounds, and oxidation reactions. Examples of endothermic reactions include dehydrogenation reactions. In particular, the reactor of the present invention may be suitable for liquid phase hydrogenation reactions (i.e. hydrogenation of liquid reactants with hydrogen vapour), for example the selective hydrogenation of butadiene to butene, the production of cyclohexane from benzene, the hydrogenation of butanal to butanol, or octanal to octanol, the hydrogenation of dimethyl succinate to 1,4-butanediol, or the production of 2-ethyl hexanol from 2-ethyl-hex-2-enal.

According to a second embodiment of the invention, there is provided a process for carrying out a gas/liquid reaction using the reactor of the first aspect. The process comprises the steps of:
(a) supplying a primary feed stream to the inlet end of the primary catalyst bed of the reactor, the primary feed stream comprising fresh feed and recycled at least partially converted liquid product;
(b) supplying a primary gas stream to the inlet end of the primary catalyst bed;
(c) allowing a reaction to occur in the primary catalyst bed;
(d) collecting an at least partially converted liquid product stream from the outlet end of the primary catalyst bed;
(e) recycling at least a portion of the at least partially converted liquid product stream to the inlet end of the primary catalyst bed;
(f) recycling at least a portion of the at least partially converted liquid product stream to the inlet end of the secondary catalyst bed of the reactor;
(g) supplying a secondary gas stream to the inlet end of the secondary catalyst bed;
(h) allowing a reaction to occur in the secondary catalyst bed; and
(i) collecting the product stream from the secondary catalyst bed, separately from the at least partially converted liquid product stream from the outlet end of the primary catalyst bed.

It will be understood that the reaction in the primary catalyst bed occurs between the primary gas stream and the primary feed stream, while the reaction in the secondary catalyst bed occurs between the secondary gas stream and the secondary feed stream.

In some embodiments, all of the at least partially converted liquid product from the primary catalyst bed is recycled. Alternatively, a portion of the at least partially converted liquid product may be collected and recovered from the reactor.

In some embodiments, the process comprises an additional step of heating or cooling the at least partially converted liquid product stream before recycling to the primary and/or secondary catalyst beds. It will be understood that the step of heating or cooling the at least partially converted liquid product stream occurs between steps (d) and (e)/(f) of the process.

In some embodiments, the process involves individually controlling the flowrate of the primary gas stream and the secondary gas stream. The flowrate of the primary and secondary gas streams may be controlled by any suitable means, for example by separate flow control valves on the respective lines.

The process may be used for carrying out any suitable reaction. In some embodiments, the reaction is hydrogenation of an aldehyde to an alcohol. Alternatively, the reaction may be selective hydrogenation of a diene or an alkyne to an olefin. In other embodiments, the reaction is hydrogenation of the aromatic ring in an aromatic compound.

The catalyst and reaction conditions used in the process will depend on the reaction being carried out. For example, where the reaction is the hydrogenation of an aldehyde, a copper/carbon or copper/chrome catalyst may be used and the reaction may be carried out at a temperature from about 140° C. to about 200° C. and a pressure of at least 1 MPa above ambient pressure. For the selective hydrogenation of dienes, a palladium or alumina catalyst may be used and the reaction may be carried out at a temperature from about 20° C. to about 130° C. and a pressure from about 0.5 MPa to about 2 MPa above ambient pressure.

### BRIEF DESCRIPTION OF THE DRAWINGS

FIG. 1 shows an example of a liquid/gas reactor known from the prior art;

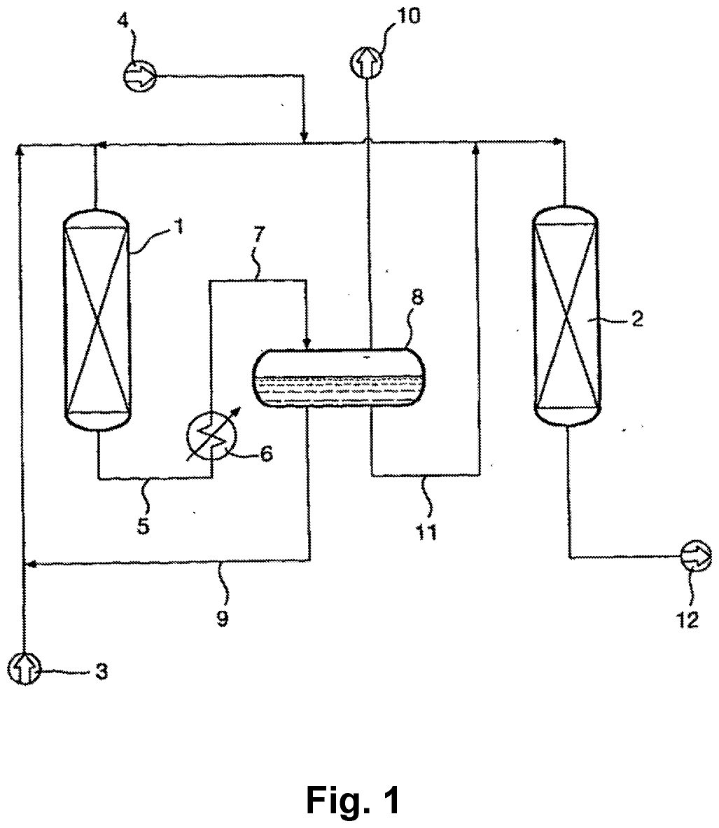

FIG. 2 shows another example of a liquid/gas reactor known from the prior art;

FIG. 3 shows a liquid/gas reactor in accordance with an embodiment of the invention;

FIG. 4 shows a liquid/gas reactor in accordance with another embodiment of the invention; and

FIG. 5 shows a close-up view of a liquid/gas reactor in accordance with an embodiment of the invention.

### DETAILED DESCRIPTION OF EMBODIMENTS

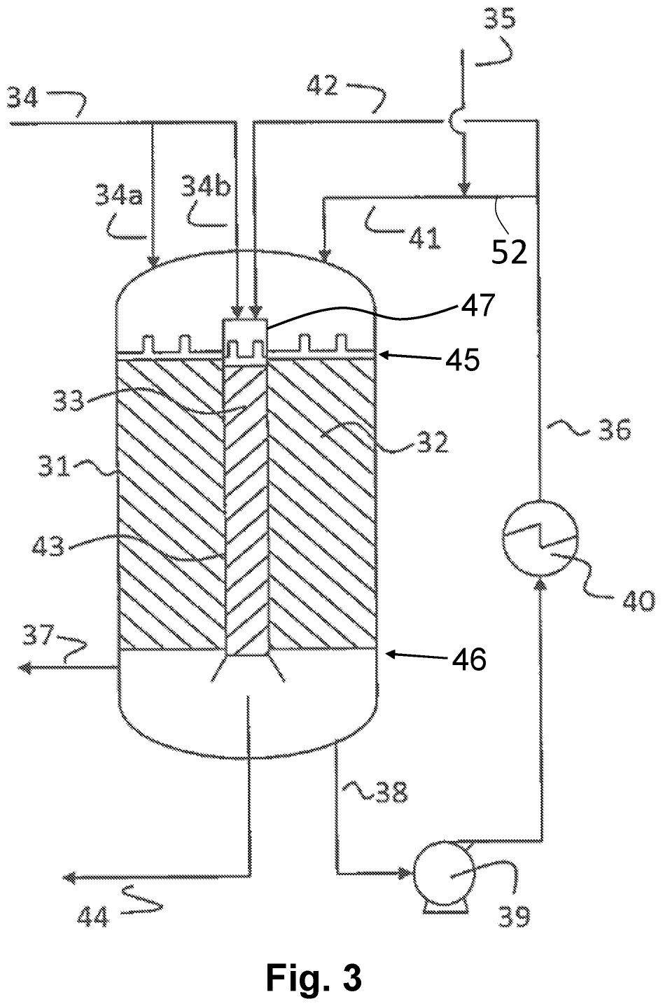

A liquid/gas reactor in accordance with an embodiment of the first aspect of the invention is illustrated schematically in FIG. 3. The reactor 31 comprises a primary catalyst bed 32 and a secondary catalyst bed 33, separated from each other by a separating wall 43. The secondary catalyst bed 33 is located centrally, such that the primary catalyst bed 32 forms an annulus around the secondary catalyst bed 33. Gas is supplied to the reactor via line 34, which is branched to provide a primary gas stream 34a to the primary catalyst bed 32 and a separate secondary gas stream 34b to the secondary catalyst bed 33. Each branch 34a, 34b may have respective means (not shown) for individually controlling the flowrate of gas to the primary and secondary catalyst beds 32, 33.

Fresh feed is supplied via line 35 and mixed with a portion 52 of recycled product stream 36 to provide a primary feed stream 41. The primary feed stream 41 is supplied to the primary catalyst bed 32, where reaction occurs between the primary feed stream 41 and the primary gas stream 34a. Another portion of recycled product stream 36 is provided to a secondary feed stream 42. The secondary feed stream 42 is supplied to the secondary catalyst bed 33, where further reaction occurs between the secondary feed stream 42 and the secondary gas stream 34b.

Off-gas is removed from the bottom of the reactor 31 via line 37. The at least partially converted product from the primary catalyst bed 32 is recovered via line 38 using pump 39. The temperature of the at least partially converted product stream is adjusted by heater/cooler 40, before being recycled to the primary and secondary feed streams 41, 42 via line 36. The more fully converted product from the secondary catalyst bed 33 is collected via line 44.

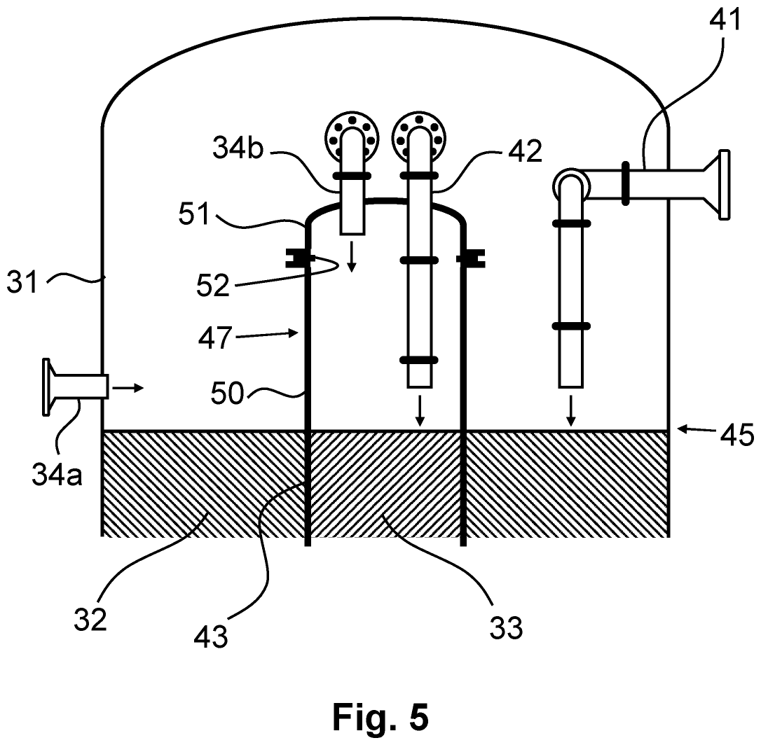

The primary and secondary catalyst beds 32, 33 have an inlet end where reactants enter (shown generally at 45), and an outlet end where products and excess reactants exit (shown generally at 46). The secondary catalyst bed 33 comprises a cover 47 (shown in more detail in FIG. 5) to isolate the inlet end of the secondary catalyst bed 33 from the inlet end of the primary catalyst bed 32. The secondary feed stream 42 and the secondary gas stream 34b are supplied to the secondary catalyst bed 33 inside the cavity defined by the cover 47, allowing a separate flowrate of gas to be supplied to the primary and secondary catalyst beds 32, 33. Without wishing to be bound by theory, it is thought that individually controlling the flowrate of gas to the primary and secondary catalyst beds allows the effect of different pressure drops through the primary and secondary catalyst beds to be mitigated, ensuring a consistent reaction rate and thus a consistent and improved overall conversion rate regardless of any differences in pressure drop.

The reactor of the present invention may be used for the hydrogenation of aliphatic C₂-C₂₀ aldehydes to the corresponding alcohol over a Cu/Cr or Cu/C catalyst. For this reaction, the same catalyst will generally be used in both catalyst beds. The residence time, based on feed, will be about 0.1 to about 10 hours. The temperature of the catalyst beds will be in the region of about 100° C. to about 200° C. and the absolute pressure will be about 0.1 to about 5 MPa. Alternatively, the reaction may be carried out over a nickel catalyst in which case the residence time, based on feed, will be about 0.1 to about 10 hours. The reaction will be carried out at temperatures from about 70° C. to about 150° C. and at absolute pressures from about 0.1 to about 5 MPa.

It is believed that recycle of the at least partially converted product stream advantageously restricts the temperature rise across the reactor. By limiting the temperature rise, the outlet temperature can be limited. This has the benefit of limiting, or avoiding, by-product formation and may provide improved selectivity. In addition, a low inlet temperature is avoided. This is beneficial, since a low inlet temperature would require a large induction zone in the reactor inlet before the reaction could start. However, the recycle rate is preferably not be larger than necessary, as this unduly dilutes the reactants with product and reduces the effectiveness of the catalyst.

Whichever catalyst system is used, the recycle rates will preferably be between about 1 to about 50 times the fresh feed rate. The catalyst beds may be sized so that the liquid superficial velocity is in a range of about 0.2 to about 20 cm/s. The hydrogen will generally be fed at quantities of approximately equal to or up to about double the stoichiometric requirement. Since the hydrogenation of aliphatic C₂-C₂₀ aldehydes is an exothermic reaction, a cooler 40 will be used to remove the heat of the reaction from the recycled product stream.

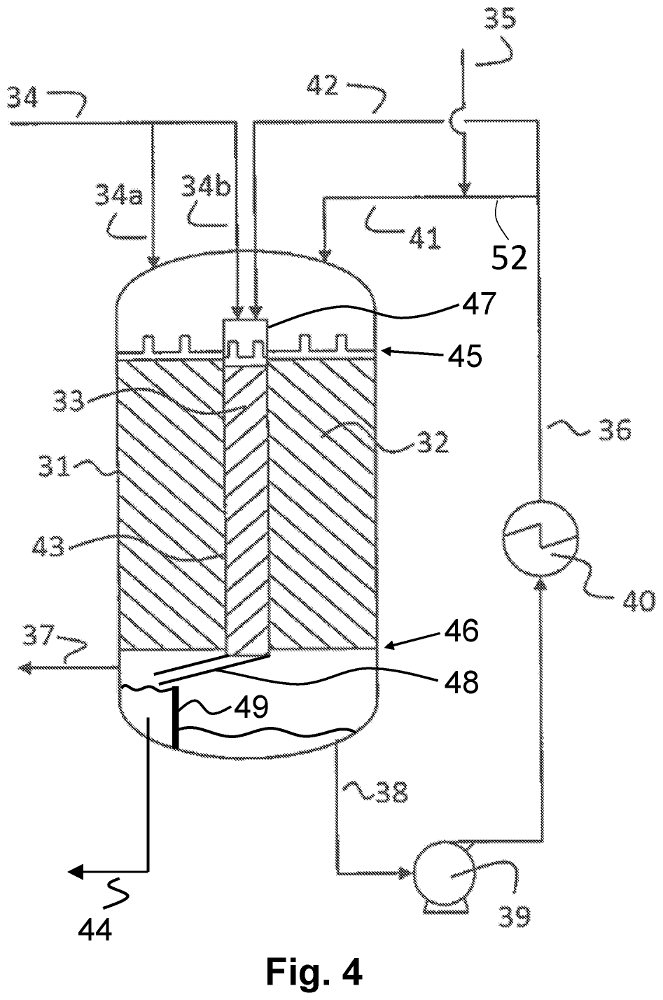

Another embodiment of the invention is illustrated schematically in FIG. 4. The reactor shown in FIG. 4 is largely the same as the reactor shown in FIG. 3, with the addition of a conduit 48 for directing the product from the outlet end of the secondary catalyst bed 33 to a weir created by baffle 49, which is offset to one side of the bottom of the reactor 31. The weir is flooded with product from the secondary catalyst bed 33, such that any overflow mixes with the partially converted product from the primary catalyst bed 32 and is recycled via line 38. The flooding of the weir with product from the secondary catalyst bed 33 may prevent entry of product from the primary catalyst bed 32. There may be a roof over the weir so that partially converted product from the primary catalyst bed 32 is deflected by the roof and doesn't enter the product recovered via line 44. The product from the weir is recovered via line 44.

A more detailed close-up view of the top of the reactor 31 is shown in FIG. 5. The secondary catalyst bed 33 comprises a cover 47, which comprises a continuous sidewall 50 formed by an extension of the separating wall 43. The cover 47 further comprises a removable cap 51, which allows access to the secondary catalyst bed 33 when required, such as for replacement of the catalyst. The cover 47 also comprises a gasket 52 for creating a gastight seal between the sidewall 50 and the cap 51. The cover defines a cavity above the inlet end of the secondary catalyst bed 33, into which the secondary gas stream 34b and secondary feed stream 42 are supplied. This keeps the secondary gas stream 34b separated from the primary gas stream 34a, which is supplied to the inlet end of the primary catalyst bed 32, and allows the flowrates of the primary and secondary gas streams 34a, 34b to be individually controlled.

### EXAMPLES

### Effect of Differing Pressure Drops on Reactor Performance

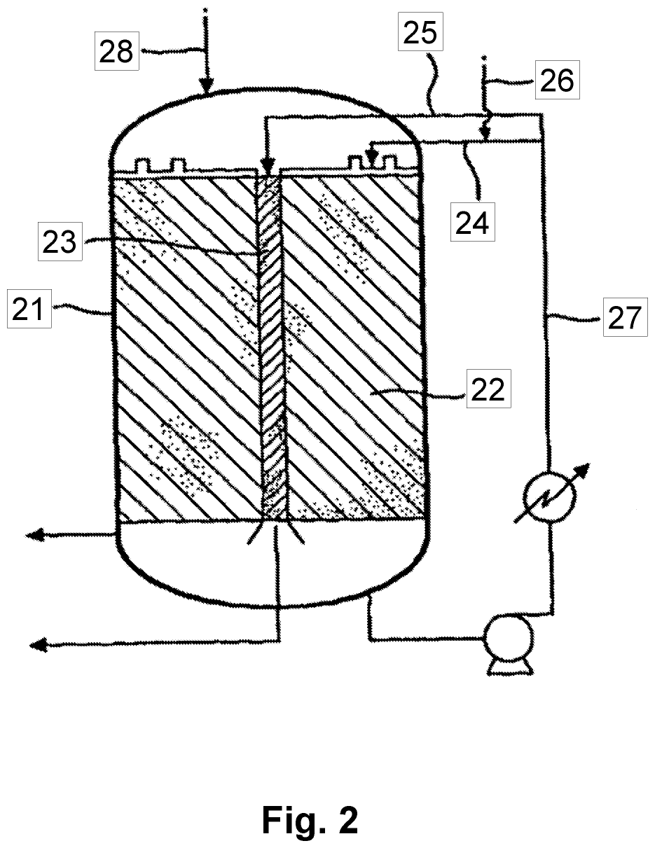

The performance of a reactor as shown in FIG. 2 was investigated with different secondary catalyst bed conditions resulting in different pressure drops. The reactor was used with a hydrogenation of butyraldehyde to butanol.

The primary and secondary catalyst beds each had the same bed length of 10,000 mm. The primary catalyst bed had a diameter of 1000 mm. The pressure at the top of the reactor was 3 MPa, while the pressure at the bottom of the reactor was largely a function of the pressure drop through the primary catalyst bed, the pressure drop being calculated by the Ergun equation. The assumed constant temperature was set at 150° C. The flow rate of hydrogen through the secondary catalyst bed was measured under different bed conditions (Examples 1-3).

### Comparative Example 1

Comparative Example 1 was a reference example. The particle size and packing in the secondary catalyst bed was the same as in the primary catalyst bed, resulting in identical pressure drops through the primary and secondary catalyst beds. The hydraulic diameter of the particles was 1.6755 mm and the void fraction was 0.38.

### Comparative Example 2

Comparative Example 2 was used to measure the effect of changing the particle size of the secondary catalyst bed while keeping the void fractions the same. The particles in the secondary catalyst bed were smaller in diameter (1.4904 mm) than the particles in the primary catalyst bed (1.6755 mm). The void fraction of both beds was the same as in Comparative Example 1 (i.e. 0.38). Smaller particles could result, for example, from undesired attrition of the particles during loading of the catalyst into the reactor. Each loading of the catalyst particles will be somewhat different, and the level of undesired attrition may therefore be different from one loading to the next.

### Comparative Example 3

Comparative Example 3 was used to measure the effect of changing the void fraction in the secondary catalyst bed while keeping the particle sizes the same. The secondary catalyst bed had a larger void fraction (0.40) than the void fraction of the primary catalyst bed (0.38). The particle size of both beds was the same as in Comparative Example 1 (i.e. 1.6755 mm). Different void fraction could occur, for example, from different compaction of the catalyst particles as they are loaded into the reactor. Each loading of the catalyst particles will be somewhat different, and the compaction may therefore alter from one loading to the next.

Table 1 shows the flow rate achieved through the secondary catalyst bed in each of Comparative Examples 1-3.

TABLE 1

| Conditions | | | | | | |
|---|---|---|---|---|---|---|
| in | | | | | | |
| secondary | Comparative | | Comparative | | Comparative | |
| catalyst | Example 1 | | Example 2 | | Example 3 | |
| bed | Gas | Liquid | Gas | Liquid | Gas | Liquid |
| Hydraulic | 1.6755 | | 1.4903 | | 1.6755 | |
| Particle | | | | | | |
| diameter | | | | | | |
| (mm) | | | | | | |
| Bed void | 0.38 | | 0.38 | | 0.40 | |
| fraction | | | | | | |
| Flow rate | 100 | 40,000 | 81 | 40,000 | 122 | 40,000 |
| (kg/h) | | | | | | |

In Comparative Example 2, the flow of hydrogen through the secondary catalyst bed was reduced, which could lead to a reduced reaction rate, resulting in reduced conversion, less effective use of the secondary catalyst and reduced reactor performance.

In Comparative Example 3, the flow of hydrogen through the secondary catalyst bed was increased, which can lead to poorer selectivity, reduced residence time and/or reduced conversion. It may also provide a route for the hydrogen to bypass the primary catalyst bed and result in less hydrogen flow going through the primary catalyst bed, leading to reduced conversion in the primary reactor bed and reduced reactor performance.

### Effect of Providing Separate Primary and Secondary Gas Streams

By contrast to the above examples, in a reactor according to the present invention, as shown in FIG. 3, the pressure drop through the primary and secondary can be independently controlled. Again, the primary and secondary catalyst beds each had the same bed length of 10,000 mm. The primary catalyst bed had a diameter of 1000 mm. The pressure at the top of the primary catalyst bed was 3 MPa, while the pressure at the bottom of the reactor was largely a function of the pressure drop through the primary catalyst bed, the pressure drop being calculated by the Ergun equation. The pressure at the top of the secondary catalyst bed was controlled so as to keep a constant flow rate through the secondary catalyst bed. The assumed constant temperature was set at 150° C.

### Example 4

Example 4 was a repeat of Comparative Example 1, but with a reactor as shown in FIG. 3 and the pressure at the top of the secondary catalyst bed controlled so as to keep a constant flow rate through the secondary catalyst bed.

### Example 5

Example 5 was a repeat of Comparative Example 2, but with a reactor as shown in FIG. 3 and the pressure at the top of the secondary catalyst bed controlled so as to keep a constant flow rate through the secondary catalyst bed.

### Example 6

Example 6 was a repeat of Comparative Example 3, but with a reactor as shown in FIG. 3 and the pressure at the top of the secondary catalyst bed controlled so as to keep a constant flow rate through the secondary catalyst bed.

Table 2 shows the flow rate achieved through the secondary catalyst bed in each of Examples 4-6.

TABLE 2

| Conditions | | | | | | |
|---|---|---|---|---|---|---|
| in | | | | | | |
| secondary | | | | | | |
| catalyst | Example 4 | | Example 5 | | Example 6 | |
| bed | Gas | Liquid | Gas | Liquid | Gas | Liquid |
| Hydraulic | 1.6755 | | 1.4903 | | 1.6755 | |
| Particle | | | | | | |
| diameter | | | | | | |
| (mm) | | | | | | |
| Bed void | 0.38 | | 0.38 | | 0.40 | |
| fraction | | | | | | |
| Flow rate | 100 | 40,000 | 100 | 40,000 | 100 | 40,000 |
| (kg/h) | | | | | | |

Because the present invention allows the pressure at the top of the secondary catalyst bed to be controlled independently so as to keep a constant flow rate through the secondary catalyst bed, the issues identified above in relation to the Comparative Examples do not occur and optimal reactor performance is maintained across all the Examples.

1. A liquid/gas reactor comprising:
(a) a primary catalyst bed having an inlet end and an outlet end;
(b) means for supplying a primary feed stream to the inlet end of the primary catalyst bed, the primary feed stream comprising fresh feed and recycled at least partially converted liquid product;
(c) a secondary catalyst bed having an inlet end and an outlet end, the secondary catalyst bed extending substantially vertically through the primary catalyst bed;
(d) means for supplying a secondary feed stream to the inlet end of the secondary catalyst bed, the secondary feed stream comprising recycled at least partially converted liquid product;
(e) means for collecting at least partially converted liquid product from the outlet end of the primary catalyst bed and recycling at least a portion of the at least partially converted liquid product to the inlet end of the primary catalyst bed and secondary catalyst bed;
(f) a separating wall between the primary catalyst bed and secondary catalyst bed;
(g) means for supplying a primary gas stream only to the inlet end of the primary catalyst bed; and
(h) means for supplying a secondary gas stream only to the inlet end of the secondary catalyst bed.

2. The reactor of claim 1, further comprising means for controlling the flowrate of the primary gas stream and the flowrate of the secondary gas stream individually.

3. The reactor of claim 1, wherein the secondary catalyst bed is located in the centre of the primary catalyst bed, such that the primary catalyst bed forms an annulus around the secondary catalyst bed.

4. The reactor of claim 1, wherein the separating wall is made from an insulating material.

5. The reactor of claim 1, wherein the secondary catalyst bed comprises a cover to isolate the inlet end of the secondary catalyst bed from the inlet end of the primary catalyst bed, and wherein the secondary gas stream and secondary feed stream are supplied to the inlet end of the secondary catalyst bed inside the cover.

6. The reactor of claim 5, wherein the cover comprises an extension of the separating wall above the primary and secondary catalyst beds.

7. The reactor of claim 5, wherein the cover comprises a removable cap.

8. The reactor of claim 7, wherein the cover further comprises a gasket for creating a gastight seal with the removable cap.

9. The reactor of claim 1, further comprising means for collecting a product stream from the outlet end of the secondary catalyst bed.

10. The reactor of claim 9, wherein the means for collecting a product stream from the outlet end of the secondary catalyst bed includes a conduit for diverting the product stream from the secondary catalyst bed to a receiving portion of the reactor, the receiving portion being isolated from the outlet end of the primary catalyst bed.

11. The reactor of claim 1, wherein all of the at least partially converted product from the outlet end of the primary catalyst bed is recycled, with a portion being recycled to the inlet end of the primary catalyst bed and a portion being recycled to inlet end of the secondary catalyst bed.

12. The reactor of claim 1, further comprising means for adjusting the temperature of the recycled stream of at least partially converted liquid product.

13. The reactor of claim 1, further comprising means for controlling the flowrate of the primary feed stream and the flowrate of the secondary feed stream individually.

14. The reactor of claim 1, wherein a feed stream ratio is the ratio of secondary feed stream flowrate to primary feed stream flowrate, wherein a bed cross-sectional area ratio is the ratio of secondary catalyst bed cross-sectional area to primary catalyst bed cross-sectional area, and wherein the bed cross-sectional area ratio is from 0.5 to 2 times the feed stream ratio.

15. The reactor of claim 14, wherein the bed cross-sectional area ratio is 1:1.

16. A process for carrying out a gas/liquid reaction using the reactor of claim 1, the process comprising the steps of:
(a) supplying a primary feed stream to the inlet end of the primary catalyst bed of the reactor, the primary feed stream comprising fresh feed and recycled at least partially converted liquid product;
(b) supplying a primary gas stream to the inlet end of the primary catalyst bed;
(c) allowing a reaction to occur in the primary catalyst bed;
(d) collecting an at least partially converted liquid product stream from the outlet end of the primary catalyst bed;
(e) recycling at least a portion of the at least partially converted liquid product stream to the primary feed stream;
(f) recycling at least a portion of the at least partially converted liquid product stream to the inlet end of the secondary catalyst bed of the reactor;
(g) supplying a secondary gas stream to the inlet end of the secondary catalyst bed;
(h) allowing a reaction to occur in the secondary catalyst bed;
(i) collecting the product stream from the secondary catalyst bed, separately from the at least partially converted liquid product stream from the outlet end of the primary catalyst bed.

17. The process of claim 16, wherein all of the at least partially converted liquid product from the primary catalyst bed is recycled.

18. The process of claim 16, comprising an additional step of heating or cooling the at least partially converted liquid product stream before recycling.

19. The process of claim 16, wherein the flowrate of the primary gas stream and the flowrate of the secondary gas stream are individually controlled.

20. The process of claim 16, wherein the reaction is hydrogenation of an aldehyde to an alcohol, selective hydrogenation of a diene or an alkyne to an olefin, or hydrogenation of the aromatic ring in an aromatic compound.
